(19) United States
(12) Patent Application Publication
(10) Pub. No.: US 2008/0161887 A1
(43) Pub. Date: Jul. 3, 2008

(54) NOBLE METAL ELECTRODES WITH NANOSTRUCTURES

(75) Inventor: Jeffrey J. Hagen, Plymouth, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Maple Grove, MN (US)

(21) Appl. No.: 11/617,108

(22) Filed: Dec. 28, 2006

(51) Int. Cl. A61N 1/05

(52) U.S. Cl. 607/72; 29/825; 607/116; 607/2

(57) ABSTRACT

An electrode assembly having homogeneous noble metal or blended alloy nanostructures for enhancement of capacitive charge injection. Applications can include stimulation of the baroreflex, neural stimulation and cardiac stimulation. In one embodiment, a matrix of substantially elongate nanocylinders or nanotubes are configured to deliver electrical charge to a target tissue, conducting charge substantially along the elongate axes of the nanostructures. The configuration enhances the real or effective area of the electrode to promote capacitive charge injection, and entraps solution for more complete recovery of electro-generated species. Certain embodiments may be configured to apply a suction through the electrode for temporary placement of the electrode for mapping the response of the electrode vs. positioning.

### BACKGROUND OF THE INVENTION

### Field of the Invention

The invention is directed generally to the field of electrodes for delivering electrical stimulus. More specifically, the invention is directed to implantable electrodes for medical devices having surface area enhancement in the form of nanostructures formed of electrically conductive materials such as noble metals.

The use of implantable electrodes to provide electrical stimulus in the treatment of medical conditions is known. Applications include, for example, heart pacing, bladder and incontinence control, and brain stimulation. The charge energy delivered by such examples are typically low, with voltages on the order of a few volts; however, the duty cycle (percentage of the time current is flowing through the electrode) is quite low. Other applications for defibrillation utilize relatively high voltage on the order of tens or hundreds of volts.

Still other electrical stimuli treatments require the delivery of voltage pulses that are on the order of several hundred millivolts or a few volts. For example, the baroreflex of the carotid sinus plays a central role in blood pressure homeostasis. Medical practitioners may have success with electrical stimulation of the baroreflex and/or carotid sinus nerves in the treatment of high blood pressure conditions that resist conventional pharmacological treatment. The stimulation voltage applied to the carotid sinus or baroreflex is in an intermediate energy regime, on the order of one to ten volts, but with a duty cycle that is one to two orders of magnitude higher than typical pacing stimuli (e.g. 20- to 100-Hz versus 1- to 2-Hz). Electrode design for such intermediate energy applications presents unique challenges, such as those discussed in U.S. Pat. No. 6,850,801 to Kieval, et al. (Kieval), which is hereby incorporated by reference herein in its entirety.

One consideration in the designed electrodes for intermediate energy applications is the means by which electrical charges are transferred from the electrode to the patient. An electrical conductor such as an electrode transfers electrical charge directly, i.e. by electron transfer. A biological medium, on the other hand, may transfer charge either directly or by ionic conduction.

The process by which charge transfer occurs at the interface of an electrode and the targeted biological medium is often characterized as being either “capacitive” or “Faradaic.” Capacitive transfer involves the attraction and repulsion of ions in solution. The ions effectively act as electron carriers to transfer charge between the electrode and the target tissue. Faradaic transfer involves the transfer of electrons from the electrode surface to species in solution. This occurs both as a removal of electrons (oxidation) at the anodic electrode and as an addition of electrons (reduction) at the cathodic electrode. Charge transfer through the tissue occurs in a similar fashion to capacitive current where positive ions migrate towards the cathodic electrode and negative electrons migrate towards the anodic electrode.

Capacitive transfer is the preferred mode of charge injection in medical applications. The process is reversible and can be tailored for minimal net change in the chemical composition of the solution medium through techniques such as biphasic pulsing. Capacitive transfer also presents a minimal alteration of the chemistry at the electrode interface, thereby mitigating biocompatibility concerns.

Faradaic transfer is typically the less desirable mode of charge injection in the context of medical electrodes. The electrochemical reactions in the Faradaic process can be irreversible and can introduce deleterious alterations in the chemical composition of the solution. The electrodes themselves may be adversely affected by formation of oxides or compounds that lead to dissolution of the electrode. Moreover, these oxides and compounds may be toxic to the organism.

In practice, transfer of electrical charge through an ionic medium generally occurs by both the capacitive and the Faradaic mechanism simultaneously. However, it is known that capacitive transfer is dominant at current densities that are below about 0.001 Amperes/mm², and that Faradaic transfer is dominant above this threshold. For a more complete discussion of capacitive and Faradaic transfer, reference is made to Ballestrasse, et al. (Ballestrasse), “Calculations of the pH changes produced in body tissue by a spherical stimulation electrode,” Annals of Biomedical Engineering, v. 13, pp. 405-24 (Pergamon Press Ltd., 1985), which is hereby incorporated by reference in its entirety. Ballestrasse has predicted minimal excursions in the pH changes of interstitial solutions for spherical contact structures that are less than about 1-μm. While Ballestrasse does not expound on the importance of pH excursions, the pH excursion is an indication of the diffusion of incomplete or irreversible electro-generated species.

Accordingly, electrodes are typically designed to have an enhanced or maximized surface area to increase the “capacitive layer”—i.e. the layer of solution in contact with the electrode. An increased capacitive layer spreads the required electron transfer over a larger surface area, thereby reducing the current density or the charge density at the electrode/solution interface. A “geometric” surface area, sometimes referred to as a “footprint,” is calculated from the overall shape and dimension of the electrode. A “real” or “effective” surface area of the same electrode will be greater than the geometric surface area, due to the roughness or other three-dimensional characteristics. Electrodes may be characterized by a “roughness factor” (RF), taken as the ratio of the “real” to the “geometric” areas. At a molecular level, the RF can be quite substantial. Even a highly polished, mirror-like platinum surface will have a RF on the order of 1.3 or 1.4. The higher the RF, the greater the area enhancement and attendant size of the capacitive layer.

Surface area enhancement also reduces the impedance of the delivery circuit, thereby enabling delivery of effective stimulation charges at lower voltages. Low impedances reduce the susceptibility of the system to shunting pathways that result from small defects in insulation and encapsulation, and also reduce exposure of neighboring tissues in the event of catastrophic circuit or package failure.

Electrode surface area for stimulation electrodes has typically been increased by either roughening the surface (mechanically or by chemical etching) or by coating the electrode with a material providing a roughened surface (e.g. titanium nitride coatings). Unfortunately, mechanical and chemical roughening of electrode surfaces are often hard to control and can produce inconsistent surface characteristics.

Recent advances in medical electrode technology include the implementation of carbon or carbon-doped nanotubes in electrode coatings, such as disclosed in U.S. patent application Publication 2005/0075708, which is hereby incorporated by reference in its entirety. The nanotube structures described in this application are intended to greatly increase the effective surface area of the electrode.

Another recent advance in the fabrication and assembly of nanostructures is a welding technique known as “nanorobotic” spot welding. The technique involves filling carbon nanotubes with copper and subjecting the filled nanotube to a current sufficient to melt the copper. The flowing copper cements the nanotube to other structures. See “Nano-Welds Herald New Era of Electronics,” NewScientist.com News Service, 19 Dec. 2006, which is hereby incorporated by reference in its entirety.

While the use of carbon and carbon-doped nanotubes to increase electrode surface area may be suitable for low energy neurostimulator applications or higher energy applications having a low duty cycle requirement, the suitability of such carbon-based nanotubes in the intermediate energy regime and at moderate or high duty cycle applications is questionable. Systems that operate in intermediate energy regimes implementing a high pulse rate duty cycle are known to be the greatest energy consumers among implantable devices. Accordingly, one concern is that a carbon based structure will break down under such duty cycle demands.

Also, the electrical resistivity of carbon nanotube structures is known to be high relative to more traditional electrode configurations. Even if the electrodes hold together, the increase in electrical resistivity requires higher voltages to deliver equivalent charges, thereby placing higher demand on finite power supplies such as batteries. Battery life is recognized as a problematic issue, and designers can ill afford to implement electrodes that require higher sustained voltages.

Furthermore, the technique of coating a metallic surface with carbon-based nanotubes or bonding carbon-based nanotubes to a base structure with a copper flow raises mechanical fatigue concerns. In a moderate or high duty cycle application, the electrode may be continuously subjected to stretch and relaxation cycles due to thermal expansion effects or natural biological processes for example. The differential expansion or movement between the nanotube coating and the base metal, coupled with the rapid accumulation of cycles, calls into question the physical stability and the fatigue characteristics of the nanotube surface coating. There are also concerns regarding the chemical/electrochemical stability of copper or certain other bonding materials or agents used to attach the nanotobes to the base electrode.

Another consideration in the design of implantable electrodes is the uniformity of the charge delivery. Generally, it is desirable to have an electrode that delivers a uniform charge over the electrode/target tissue interface. However, the quest for low profile electrodes has driven toward designs that have low aspect ratios (i.e. the ratio of the thickness to a characteristic length such as the diameter), as well as delivery of charges to the electrode that is typically introduced at or near one edge the electrode. The resulting charge distribution over the body of the electrode during the life of a transient pulse may suffer from non-uniformities due to the high resistance-capacitance (RC) product that is attendant to low profile designs.

An additional consideration in the design of electrodes is the diffusion of the ionic transfer medium. The reversibility of the capacitive transfer mechanism relies on the ions remaining within an interstitial region bounded by the electrode and the target tissue. Ions that remain in this region can be regenerated by techniques such as biphasic pulsing, resulting in minimal net change in the ion population. However, ions that diffuse away from the interstitial region before they can be restored may cause biological complications for a patient if their population becomes excessive.

Furthermore, for a given application, there is a finite limit to the charge that can be transferred reversibly in either an anodic or cathodic direction. When this limitation reached, the capacitance of the solution at the electrode interface becomes fully charged and the Faradaic reaction becomes dominant with attendant reaction products. Thus, even though designers strive to attain low charge densities and low current densities to attain capacitive transfer, most charge transfer occurs by the Faradaic mechanism, particularly in intermediate or high energy applications. It is therefore desirable for an electrode to capture or otherwise inhibit diffusion away from the interstitial layer, thereby limiting the release of ions and Faradaic reaction products into solution.

It would be desirable to provide a design for an electrode for intermediate level electro stimulation at moderate to high duty cycles that overcome the disadvantages of the existing designs and better meet the objectives for an optimum electrode tissue interface.

### BRIEF SUMMARY OF THE INVENTION

Various embodiments of the invention provide an electrode with a high effective surface area. In one embodiment, the electrode includes an arrangement of noble metal (aka “inert metal”) nanostructures disposed on an electrically conductive base suitable for conducting electrical stimulus of intermediate energy densities at moderate to high duty cycles. The high effective surface area provides a larger capacitive layer at the interface between the electrode and the target region for a given electrode size, thus enabling delivery of higher levels of charge to the target tissue while mitigating concerns regarding electrode dissolution and reducing the impedance at the electrode-tissue interface for efficient transfer of electrical charge.

The electrode may be configured to inhibit diffusion of the solution ions away from the capacitive layer, thereby enabling recovery of the electro-generated species during charge recovery phases. Such recovery reduces damage to the tissue in the target region.

In one embodiment, the invention may also be configured to provide a suction force for trial placement of the electrode against the target tissue. This aspect of the invention enables temporary or trial placement of the electrode for optimization or mapping of homeostatic response to electrical stimulation versus the location of the electrode.

Certain embodiments are comprised of conductive nanostructure materials that provide the chemical and electrochemical stability necessary to deliver moderate to high energy and frequency electrical pulses that would cause standard carbon-based structures to break down. The invention may be configured to capitalize on the advantages offered by nanostructures without the adverse effects of cyclic expansion and contraction associated with carbon nanostructures. Moreover, the materials selection for these embodiments are believed to possess lower resistivity than carbon-based structures, leading to less resistive loss and attendant Joule heating near the target tissue.

In one embodiment, a plurality of inert metal nanostructures such as nanotubes or nanowires are placed in contact with a targeted tissue region to enhance or increase the effective surface area through which electrical stimulus is delivered to the target region. The nanostructures are typically of an elongate structure such as a cylinder or a tube. Surface area enhancement is provided by the flow of tissue and solution ions into the voids between adjacent nanostructures. In one embodiment, the arrangement of nanostructures is configured as a matrix that may be densely packed to limit the diffusion of electro-generated species away from the interstitial capacitive layer. In some embodiments, the nanostructures may be fabricated from the same material as the base electrode.

In another embodiment, the nanostructures are of a hollow or cavernous construction (e.g. nanotubes), such that each defines a nanochamber. The surfaces of the interior chambers provide additional area for contact with tissue and solution ions. Moreover, the nanochambers may capture the electro-generated species at the capacitive layer for further recovery of the electro-generated species during charge recovery phases.

Some of the various embodiments may comprise nanostructures that are substantially bundled in a matrix so that the major lengths of the nanostructures are parallel with respect to each other in a closely spaced arrangement. Stimulus electricity is conducted substantially along the major lengths of the nanostructures.

In those embodiments where a fraction of the electrode is comprised of elongated void volumes, the electrode nanostructures may be configured to enable a suction to be applied through these elongated void volumes to the electrode/tissue interface, thereby providing a mechanism for increasing adherence of the electrode to the target tissue.

The inert metal nanostructures are typically comprised of a single noble metal material such as, but not limited to, gold, silver, platinum, palladium, rhodium or iridium. Alternatively, noble metal materials may be mixed with ruthenium or other base metals to achieve desired mechanical or electrical characteristics and in such a way that the blended alloy is also biocompatible.

The use of noble metals also enables attachment of the nanostructures to the base electrode by fusion techniques such as laser welding or nanorobotic spot welding.

### BRIEF DESCRIPTION OF THE DRAWINGS

FIG. 1 is a perspective view of an electrode assembly in an embodiment of the invention;

FIG. 2 is a perspective view of an arrangement of nanostructures comprising nanocylinders in the embodiment of FIG. 1;

FIG. 3 is a perspective view of an arrangement of nanostructures comprising nanotubes in the embodiment of FIG. 1;

FIG. 4 is a perspective, partial cutaway view of an electrode assembly in an embodiment of the invention;

FIG. 4A depicts the embodiment of FIG. 4 with a suction manifold attached thereto;

FIG. 5 is a perspective view of a partitioned electrode assembly in an embodiment of the invention;

FIGS. 6A, 6B and 6C portray the charge distribution at the distal faces of the electrode embodiments of FIGS. 1, 4 and 5 respectively.

FIG. 7 is a perspective view of a segment of a non-uniform matrix of nanostructures according to the invention;

FIG. 8 is a partial cross-sectional view of the non-uniform matrix of FIG. 7;

FIG. 9 is a cross-sectional view of an electrode having a matrix of randomly oriented nanostructures in an embodiment of the invention;

FIG. 10 is a schematic representation of a charge injection system according to an embodiment of the invention;

FIG. 11 is a cross-sectional view of a nanocylinder nanostructure applied to a target tissue in accordance with the present invention;

FIG. 12 is a cross-sectional view of a nanotube nanostructure applied to a target tissue in accordance with the present invention;

FIG. 13 is a plan view of an assembly for wrapping embodiments of the invention about a carotid sinus; and

FIG. 14 is a perspective view of the assembly of FIG. 13 applied to a carotid sinus.

### DETAILED DESCRIPTION OF THE INVENTION

References to relative terms such as upper and lower, front and back, left and right, or the like, are intended for convenience of description and are not contemplated to limit the present invention, or its components, to any specific orientation. All dimensions depicted in the figures may vary with a potential design and the intended use of a specific embodiment of this invention without departing from the scope thereof.

Each of the additional figures and methods disclosed herein may be used separately, or in conjunction with other features and methods, to provide improved systems and methods for making and using the same. Therefore, combinations of features and methods disclosed herein may not be necessary to practice the invention in its broadest sense and are instead disclosed merely to particularly describe representative and preferred embodiments of the instant invention.

It will be understood that the present invention may be applicable to any kinds of electromagnetic stimulation of tissue. Such applications can include stimulation of the baroreflex, neural stimulation and cardiac stimulation. In certain embodiments, the present invention is particularly adapted for use with stimulation of tissue where the stimulation energy is within an intermediate range, either due to the voltages or currents of the stimulation pulses or due to the duty cycle at which the stimulation pulses are applied.

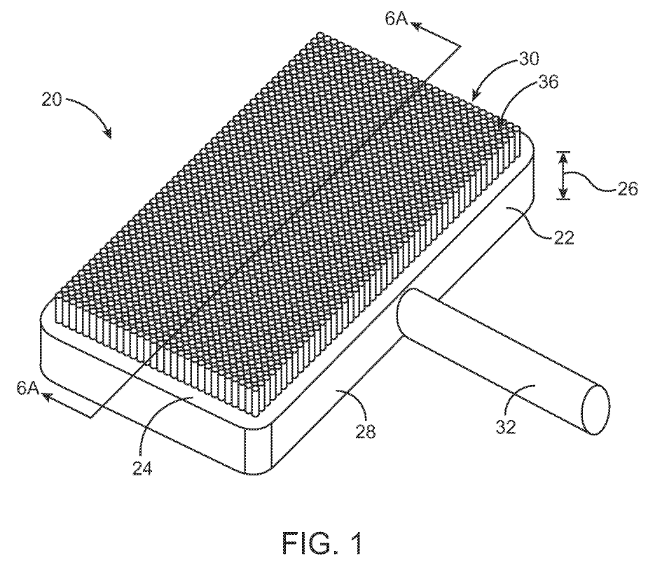

Referring to FIG. 1, an embodiment of an electrode assembly 20 according to the invention is depicted. The electrode assembly 20 includes a base 22 having a transfer surface 24, a thickness 26 and a peripheral portion 28. A plurality of nanostructures 30 are in electrical contact with and cover at least a portion of the transfer surface 24. A conductor 32 is in electrical contact with the peripheral portion 28.

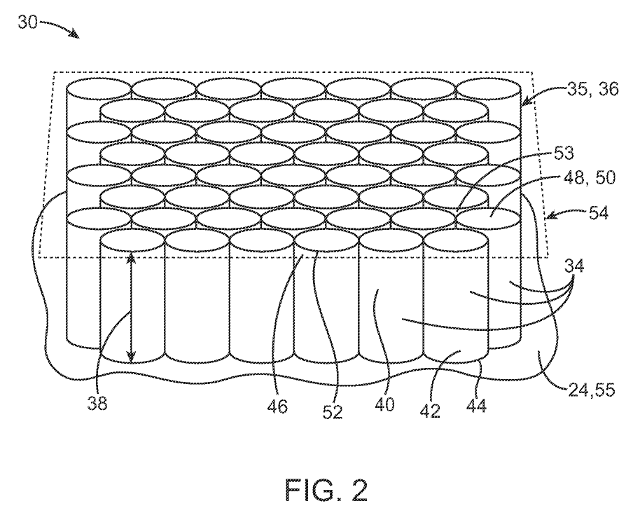

Referring to FIG. 2, an example embodiment of the nanostructures 30 is described. In this embodiment, the nanostructures comprise a multiplicity of nanowhiskers or nanocylinders 34 densely packed to form a body 35 comprising a matrix 36. Each nanocylinder 34 is characterized by a length 38, an outer peripheral surface 40, a proximal end portion 42 having a proximal end 44, a distal end portion 46 having a distal end 48, and a cross-section 50 that defines an outer peripheral length 52. In one embodiment, the matrix 36 contains not only solid conducting nanostructures 30, but also a plurality of elongate void volumes 53 formed between the nanostructures 30.

The proximal end 44 of each nanocylinder 34 is in electrical contact with the transfer surface 24 of the base 22 of the electrode assembly 20. The distal ends 48 of the nanocylinders 34 may substantially define a distal side 54; likewise, the proximal ends 44 of the nanocylinders 34 may substantially define a proximal side 55 that is substantially parallel to the distal side 54. The nanocylinders 34 are substantially solid, and may be in tangential contact with each other. Although these embodiments are described in a planar relationship, it will be understood that the present invention may be configured on a variety of surface shapes such as circular, tubular, irregular and that the surface shapes may be either fixed or flexible.

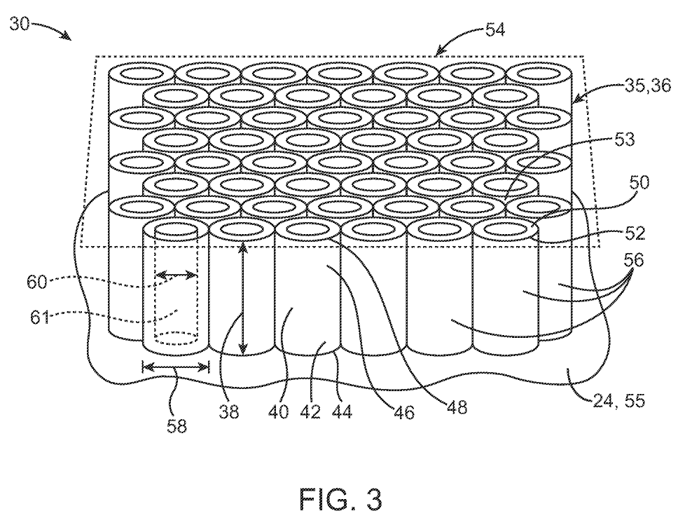

Referring to FIG. 3, another example embodiment of the nanostructures 30 is depicted comprising a multiplicity of nanotubes 56. Each nanotube is characterized by an outer diameter 58 and an inner diameter 60. Each nanotube 56 effectively creates a nanochamber 61 bounded by the inner diameter 60. In one embodiment, the nanochambers 61 are in parallel with the void volumes 53 of a comparable configuration of nanocylinders 34 (FIG. 2). Thus, the voids of the FIG. 3 configuration comprise not only the void volumes 53 between the nanostructures 30, but also the voids within the nanostructures 30.

In this embodiment, the nanotubes 56 typically possess the same characteristics as the nanocylinder embodiment of FIG. 2—i.e. a length 38, an outer peripheral surface 40, a proximal end portion 42 having a proximal end 44, a distal end portion 46 having a distal end 48, and a cross-section 50 that defines an outer peripheral length 52.

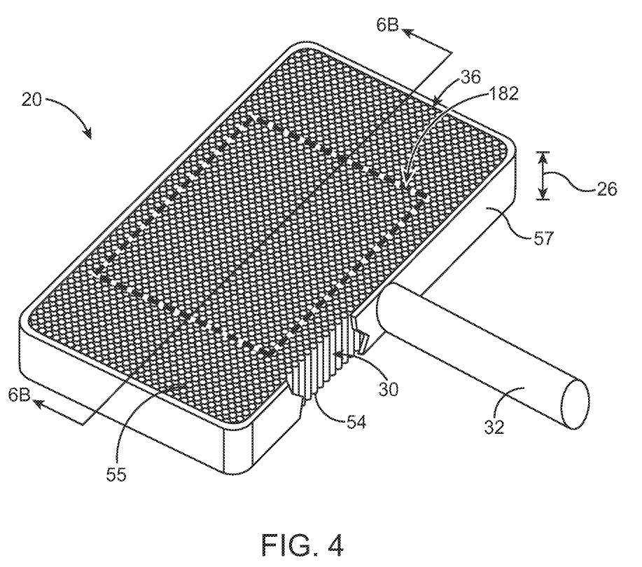

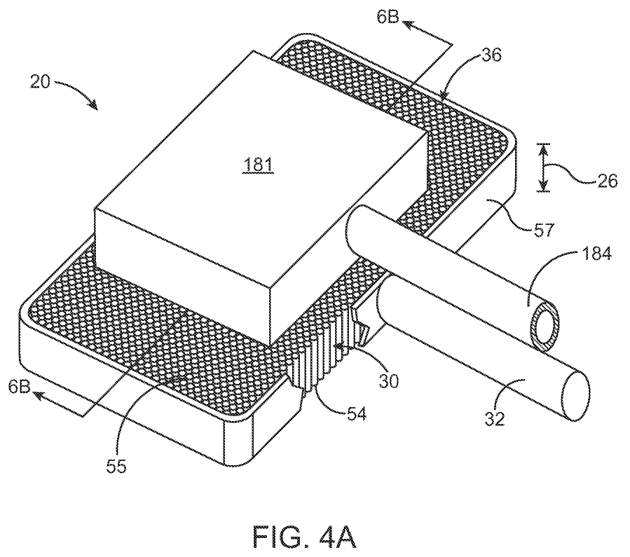

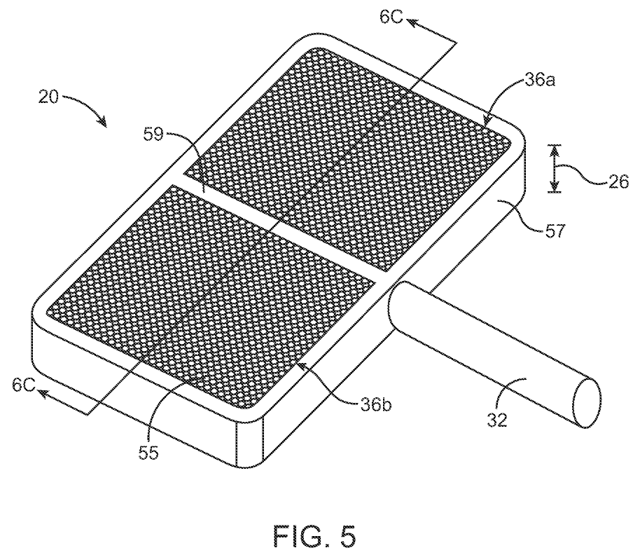

Referring to FIGS. 4, 4A and 5, another embodiment of the present invention is depicted wherein the matrix 36 of nanostructures 30 are grouped without benefit of a base portion. Rather, each of the nanostructures 30 are bonded or fused to at least one of the nanostructures adjacent to it. The nanostructures 30 may be bound on the perimeter by a skirt 57. A partition 59 may also be utilized to divide matrix 36 into segments 36a and 36b, as depicted in FIG. 5. The partition may be in intimate electrical contact with matrix segments 36a and 36b. Also, additional partitions may be implemented in a variety of orientations to further divide the matrix 36 into still smaller segments (not depicted).

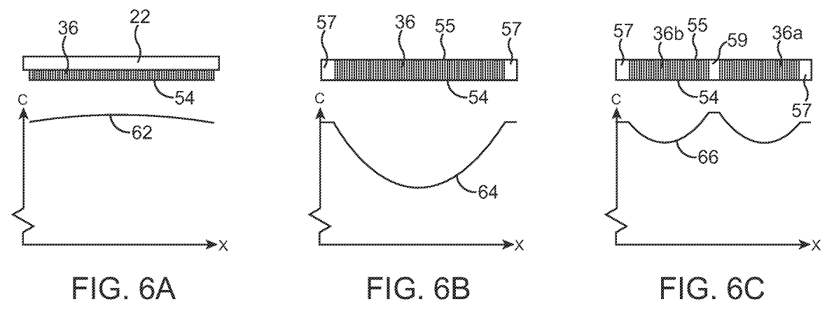

Referring to FIGS. 6A, 6B and 6C, a plurality of charge distributions 62, 64 and 66 are portrayed, one for each of the foregoing embodiments. Each distribution 62, 64 and 66 qualitatively represents the charge C along a spatial coordinate X at the face of the distal side 54 of the respective embodiment. The FIGS. 6A, 6B and 6C are anticipatory representations of the charge distributions, and are not based on actual measurements or modeling results.

In operation, the charge distribution 62 of FIG. 6A, which corresponds to the embodiment of FIG. 1, is the most uniform. The base 22, being comprised of a solid material, has a substantially lower electrical resistance than the void-filled matrix 36 of the body 35. Hence, matrix 36 acts to throttle the electrons flowing into the base 22, causing the electrons to distribute in a relatively uniform manner throughout the base 22 before passing through the matrix 36 to the distal side 54.

The charge distribution 64 depicted in FIG. 6B, anticipatory of the embodiment of FIG. 4, is the least uniform of the embodiments presented above. Electrons readily flow about the perimeter of the electrode assembly 20 via the skirt 57. However, the path to the center of the body 35 includes edge contacts between nanostructures 30 of the matrix 36, and via the thin weld layer near the proximal side 55. The electrical resistance between the perimeter and the center of the body 35 will be even greater where the matrix 36 implements non-uniform nanostructures (discussed below), i.e. where edge contacts are typically limited to point or nearly point contact. Nevertheless, the FIG. 4 configuration may still find utility in applications that can tolerate the attendant non-uniformities, or where higher peripheral charges are desirable.

A benefit of the partitioned electrode of the FIG. 5 embodiment is an expected reduction in the variation of the charge distribution 66 across the distal side 54 of the electrode assembly 20 relative to the variation of the charge distribution 64 as shown in FIG. 6C. Electrons not only flow freely within the skirt 57, but also within the partition 59. The resistive path to the central regions of the matrix segments 36a or 36b is thereby reduced, resulting in the reduced variation. The FIG. 5 embodiment is useful where it is desired to leave void volumes 53 and nanochambers 61 open from both the distal side 54 and the proximal side 55 while mitigating the effects of charge variations.

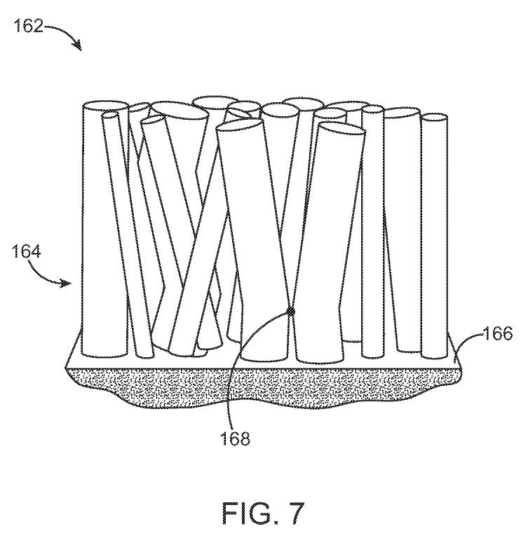

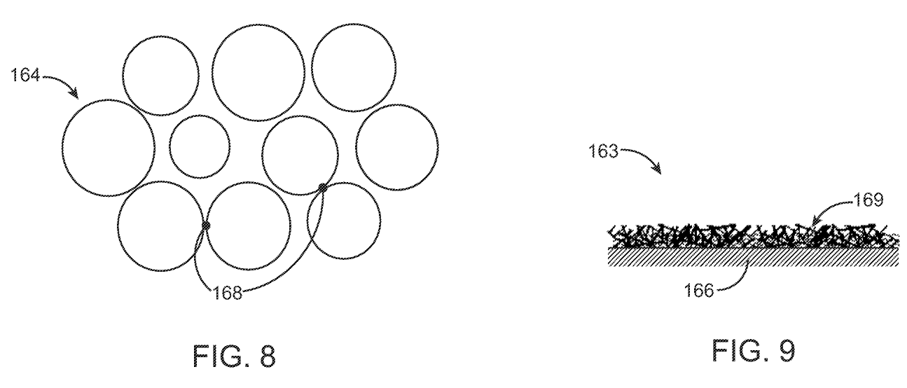

Referring to FIGS. 7 and 8, a matrix 162 of non-uniform nanostructures 164 is depicted. Referring back to FIGS. 1 through 4, the matrix 36 is depicted as comprised of right cylindrical nanostructures of uniform length 38 and uniform cross-section 50. In practice, the tolerances of nanostructures may not be enabling such uniform characteristics. As portrayed in FIGS. 7 and 8, the non-uniform nanostructures 164 may be of varying cross-section, spacing, shape and angular orientation and still function according to the spirit of the invention. The type of nanostructures portrayed in FIGS. 7 and 8 are nanocylinders; however, the non-uniform nanostructure 164 may be comprised of any variety of nanostructures such as nanobars, nano I-beams or the like.

The depiction of FIG. 7 portrays the non-uniform nanostructures 164 as being in contact with a base portion 166. However, the base portion 166 is not a necessary component to hold the matrix 162 together. It is sufficient that each nanostructure be fused with a neighboring element at only one point along its length, as depicted at numerical references 168 in FIGS. 7 and 8. The contact points 168 need not be on the same plane.

Referring to FIG. 9, a random matrix 163 comprised of a plurality of randomly oriented nanostructures 169 is illustrated. The random matrix 163 may be characterized as a nanostructure coating that covers the base portion 166.

As a practical matter, the void volumes 53 and the nanochambers 61 should be large enough to allow transport of solution ions (e.g. Na⁺, K⁺ and Cl⁻, having radii of 0.45-, 0.30- and 0.30-nm, respectively), thereby enabling the formation of an electrode/solution double layer within the void volumes 53 and the nanochambers 61. The void volumes 53 and the nanochambers 61 should therefore allow passage of ions on the order of 1-nm diameter and higher. U.S. patent application Publication 2005/0229744 by Kijima (Kijima), which is hereby incorporated by reference herein in its entirety, reports the fabrication of nanotubes with inner diameters down to 2-nm, which is large enough to satisfy the criterion.

A guideline for the maximum dimension (i.e. diameters) of a given nanostructure is less definitive. Ballestrasse's calculation of a minimal pH excursion for electrode contacts approximated by spheres on the order of 1-μm or less perhaps offers a starting point. Kijima reports several nanostructures in the art that are well below 1-μm characteristic diameter. Nevertheless, the 1-μm diameter is not to be construed as limiting for the present invention.

The nanostructures 30 or 164 may also be characterized by the outer peripheral length 52 of the cross-section 50. For example, a nanostructure having a circular cross-section with a diameter D of 1-μm will have an outer peripheral length 52 of πD, or slightly more than 3-μm at the location of cross-section 50. As the cross-section 50 departs from the circular geometry, the outer peripheral length will tend to increase. The outer peripheral length 52 provides a reasonable metric for characterizing a nanostructure because area enhancement is driven primarily by the peripheral area of the nanostructures employed.

The nanostructures 30 or 164 of the various embodiments depicted in FIGS. 1 through 9 may be bonded together by a process of laser welding. By exposing the matrix 36 or 162 to a laser of appropriate intensity, neighboring nanostructures that are in contact may locally flow together causing a bond therebetween. Generally, the proximal side 55 is subject to the laser irradiation, thereby limiting distortion of the nanostructure geometry at the distal side 54 and creating a fusion bond at a location further removed from the electrode/target tissue interface. The conductor 32 may also be joined to the matrix 36 or 162 by laser welding.

An inventive welding technique of the instant disclosure that may be implemented in fabricating certain embodiments of the invention is nanorobotic spot welding. Nanotubes may be formed from a first noble metal or biocompatible blended alloy and filled with a second noble metal or biocompatible blended alloy having a lower melting point than the first noble metal or blended alloy, with an electrical current then passed through the filled nanostructure sufficient to cause the second noble metal or blended alloy to flow. For example, iridium has a melting point of approximately 2400° C., while platinum has a melting point of approximately 1700° C. An iridium nanotube could be filled with platinum and subjected to an electrical current sufficient to raise the temperature of the composite assembly to, say, 1850° C. At this temperature, the platinum should flow sufficiently to form a bond while the structural integrity of the iridium nanotube remains intact. In an alternate embodiment, nanotubes of carbon or other biocompatible materials may have at least a portion of the interior volume of the tube filled with a noble metal or blended alloy that would then be processed by cause the filled noble metal material to melt. It may be desirable to treat or process any non-noble metal nanostructures in accordance with this embodiment in such a way as to reduce any long-term biocompatibility issues associated with the potential breakdown of such materials, particularly in response to intermediate or higher stimulation energies.

Furthermore, the nanorobotic spot weld technique, as applied to certain embodiments of the invention, does not necessitate utilizing nanochambers 61 of nanotubes 56. Consider the matrix 36 defining the elongate void volumes 53 (e.g. FIG. 3). The matrix 36 may be packed so that the second, lower melting point material resides in the elongate volumes 53 and not the nanochambers 61.

The process of heating a filler material need not be limited to joule heating by passage of electrical current. The filled nanochambers 61 or packed matrix 36 may be brought to an elevated temperature by means other than joule heating, such as by laser or microwave irradiation.

The random matrix 163 may retain many of the advantages of the uniform and non-uniform matrix configurations 36 and 162. For example, the randomly oriented nanostructures 169 will still serve to enhance the contact area over which they are coated. The randomly oriented nanostructures 169 may be packed dense enough to emulate a porous body through which a suction can be drawn while providing a substantial contact interface. A dense packing of the random matrix 163 may also provide sufficient contact between the nanostructures to enable the random matrix 163 to be fused together without need of the base portion 166.

The foregoing process enables construction of the electrode assembly 20—base 28 and/or skirt 57, partition 59 (if applicable), nanostructures 30, 164 or 169, and conductor 32—from a single material. The homogeneity of the structure precludes the differential effects caused by the bonding or joining of dissimilar materials. The process also eliminates the need for bonding materials that may introduce chemical/electrochemical concerns.

Furthermore, the connection between the nanostructures and the base or lead body may be made at a location where structural mechanics are more stable and material fracture is less likely. For example, by bonding or fusing the nanostructures on the proximal side 55 of the assembly, away from the electrode/target tissue interface, the mechanical forces experienced by the electrode assembly 20 will be partially absorbed by elastic flexure of individual nanostructures 30, 164 or 169, thereby reducing stresses at the weld joints near the proximal side 55. Accordingly, the resulting structure is more suited for higher duty and higher energy applications than are existing nanostructure electrodes.

The components of the electrode assembly 20 may thus be comprised of a single metallic material such as, but not limited to, the so-called “noble” or “inert” metals including gold, silver, platinum, palladium, rhodium or iridium. Alternatively, noble metal materials may be alloyed or “blended” with ruthenium or other base metals to achieve desired mechanical or electrical characteristics consistent with the biocompatibility required for such electrode. Another blended alloy material is a platinum-iridium (Pt—Ir) alloy. Pure platinum is generally regarded as a soft and malleable material that does not retain a defined shape under certain mechanical load scenarios. Iridium, on the other hand, is generally regarded as a brittle material and is unsuitable in certain situations where flexibility is desired. The Pt—Ir alloys possess preferable mechanical qualities for many applications. Another blended alloy family is the nickel/chromium alloys, such as MP35N, a registered trademark of SPS Technologies, Inc., of Jenkintown, Pa., U.S.A. Other biocompatible blended alloys may be utilized in certain embodiments without departing from the spirit of the invention.

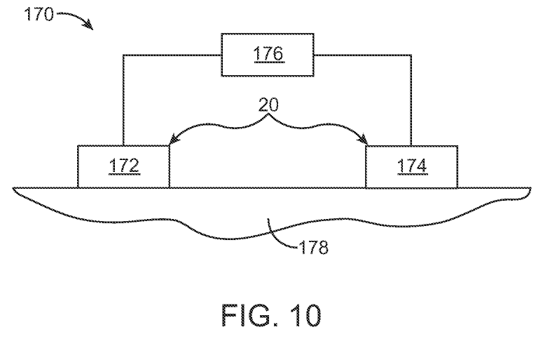

Referring to FIGS. 10 through 12, a plurality of electrodes 20 are utilized in a charge injection system 170. The charge injection system 170 comprises at least one electrode 20 designated as an anode 172, at least one electrode 20 designated as a cathode 174, the anode(s) 172 and cathode(s) 174 being in electrical communication with a charge source 176. The charge source 176 may be a voltage source or a current source. The anode(s) 172 and cathode(s) 174 are placed in substantial contact with a target tissue 178.

The particular embodiment depicted in FIG. 10 utilizes a plurality of lead lines 179 for transferring charge between the charge source 176 and the anode(s) 172 and the cathode(s) 174. It is also contemplated that charge transfer between the charge source 176 and the electrodes 20 may be induced remotely in the present invention, such as, for example, described in U.S. Pat. No. 6,061,596 and U.S. patent application Publication 2003/0158584, both of which are hereby incorporated by reference herein in their entirety.

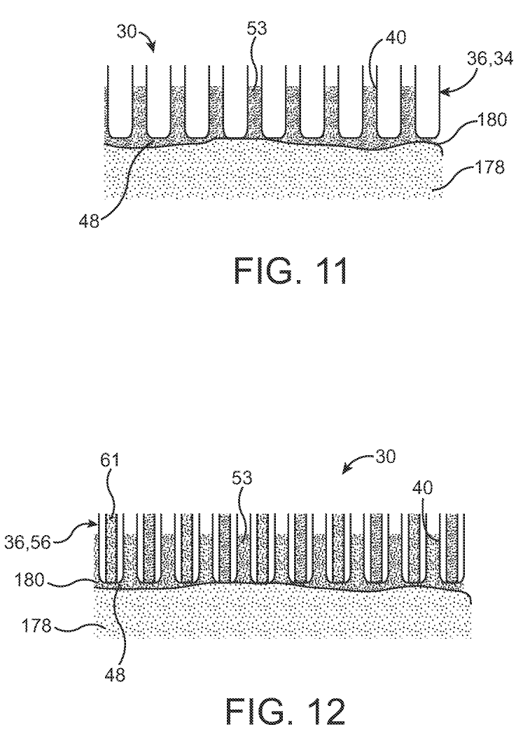

Functionally, the various embodiments act to enhance the surface area of the electrode 20, best illustrated in FIGS. 11 and 12. For example, when the matrix 36 of nanocylinders 34 is placed in contact with the target tissue 178, there will typically be a solution 180 located interstitially between the target tissue 178 and the matrix 36 (FIG. 11). The solution 180 contacts the distal ends 48 of the nanocylinders 34 and also flows into the void volume 53 and contacts the outer peripheral surfaces 40 of the nanocylinders 34.

The enhancement is further increased for hollow or cavernous nanostructures, such as when nanotubes 56 are used (FIG. 12). The solution 180 not only wets the distal ends 48 and outer peripheral surfaces 40, but also the nanochambers 61 defined by the inner diameters 60 of the nanotubes 56. All of the surfaces wetted by the solution 180 in FIGS. 11 and 12 provide an exchange surface for capacitive transfer.

Furthermore, the void volumes 53 and nanochambers 61 of the various embodiments provide a means for capturing the solution 180, which in turn promotes a more complete recovery of electro-generated species such as H⁺ and OH⁻. The capture of the solution 180 also limits the amount of the solution 180 susceptible to irreversible chemical reactions as well as the diffusion of the attendant products away from the target tissue 178, thereby limiting the undesirable effects of Faradaic transfer that may occur.

The various embodiments disclosed herein may also be configured to receive a suction means. For example, a suction manifold 181 may be placed over at least a portion 182 of the proximal side 55 of the embodiment of FIG. 4, as depicted in FIG. 4A. A suction force may be applied to the manifold 181 through a tube 184, and transferred through the void volume 53 and nanochambers 61 to draw the target tissue onto the distal side 54 of the electrode assembly 20 and attach the electrode assembly 20 to the target tissue 178. The skirt 57, which is depicted in FIGS. 4 and 4A but may be utilized in any of the disclosed embodiments, may augment the efficiency of a suction configuration.

Accordingly, filling the nanochambers 61 of the nanotubes 56 with a lower melting point noble metal or alloy blend in a nanorobotic spot weld application does not preclude the use of the matrix 36 for suction applications. Essentially, the nanorobotic spot weld process converts a nanotube structure into a nanowisker or nanocylinder structure, and suction may still be transferred through the elongate void volumes 53.

Where a packed matrix of nanotubes is utilized, the packing may be done in a way that only the elongate void volumes 53 are filled. The final assembly would thus feature clear nanochambers 61 suitable for suction configurations.

The nanorobotic spot weld technique may also be utilized in the random matrix 136 configuration. Where the nanochambers 61 of the nanotubes 56 are packed with the filler material, the material may be heated to a temperature such that the filler material flows freely from the nanochambers 61 and form liquidous contacts between adjacent nanostructures and/or the base 28 (when utilized). Upon cooling, the liquidous contacts harden and form a bond between the elements contacted.

The elongate void volumes 53 in a nanowisker or nanocylinder assembly could also be utilized to hold melting material. However, such a configuration may preclude application of a suction.

The utilization of the base 22 in configurations such as depicted in FIG. 1 does not preclude a suction configuration. The base 22 may be configured to be porous or otherwise have passages that allow application of suction to the proximal side 55 of the nanostructures 30. Also, the suction means may be applied at or about the periphery of the matrix 36 or 162, between the distal and proximal sides 54 and 55, to affect the suction. Furthermore, application of the suction placement technique is not limited to electrodes implementing nanostructures. Any electrode having a porosity that enables fluid communication between the distal side 54 that is in contact with the target tissue 178 and the body 35 of the electrode, and is configured to apply a suction to the distal side 54, is suitable for the application.

In operation, the suction force may be used for temporary placement of the electrode for mapping the response of the organism versus electrode placement. The suction provides a rapid, easily reversible means for operably connecting the anode(s) 172 and cathode(s) 174 to the target tissue 178.

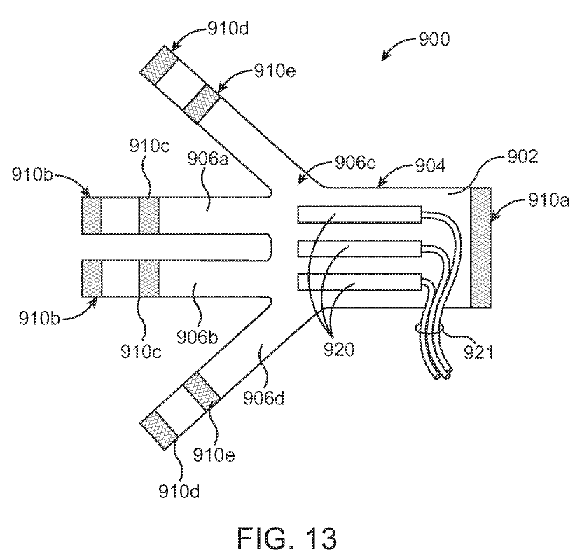

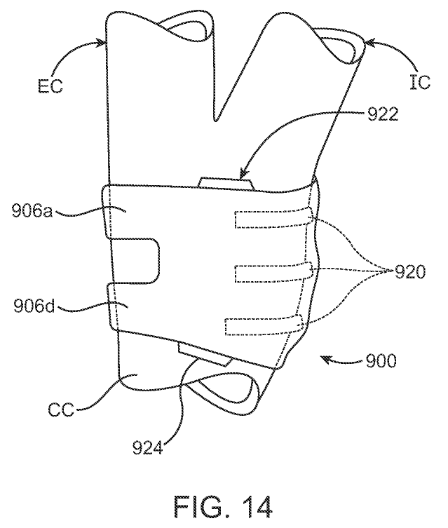

Referring to FIGS. 13 and 14, a wrap assembly 900 implementing aspects of the present invention for application in the stimulation of a carotid sinus is presented. The wrap assembly 900 comprises base wrapping 902 that is typically formed from silicone or other elastomeric material, and having an electrode carrying surface 904 and a plurality of attachment tabs 906 (906a, 906b, 906c, and 906d) extending from the electrode carrying surface.

The geometry of the wrap assembly 900, and in particular the geometry of the base 902, is selected to permit a number of different attachment modes to the blood vessel. In particular, the geometry of the base wrapping 902 of FIG. 13 is intended to permit attachment to various locations on the carotid arteries at or near the carotid sinus and carotid bifurcation.

A number of reinforcement regions 910 (910a, 910b, 910c, 910d, and 910e) are attached to different locations on the base 902 to permit suturing, clipping, stapling, or other fastening of the attachment tabs 906 to each other and/or the electrode-carrying surface 904 of the base wrapping 902. In the preferred embodiment intended for attachment at or around the carotid sinus, a first reinforcement strip 910a is provided over an end of the base 902 opposite to the end which carries the attachment tabs. The pairs of reinforcement strips 910b and 910c are provided on each of the axially aligned attachment tabs 906a and 906b, while similar pairs of reinforcement strips 910d and 910e are provided on each of the transversely angled attachment tabs 906c and 906d. In the illustrated embodiment, all attachment tabs are provided on one side of the base wrapping 902, preferably emanating from adjacent corners of the rectangular electrode-carrying surface 904.

The structure of the wrap assembly 900 enables the surgeon to implant the electrode assembly so that one or more nanostructure electrode assemblies 920 are in contact with the carotid artery and are positioned for stimulation of tissue therein. The electrode assemblies 920 each include an electrically insulated lead 921 that may be constructed in accordance with the embodiments disclosed herein, or other embodiments that implement aspects of the invention. The preferred location may be determined, for example, by the temporary suction method described above, or by other methods such as that described in Kieval.

Once the preferred location for the nanostructure electrodes 920 of the wrap assembly 900 is determined, the surgeon may position the wrapping base 902 so that the nanostructure electrodes 920 are located appropriately relative to the underlying tissue. Thus, the nanostructure electrodes 920 may be positioned over a portion of the carotid sinus such as the common carotid artery CC, as depicted in FIG. 14, or placed over the internal carotid artery IC or the external carotid artery EC (not depicted). The wrap assembly 900 may be attached by stretching the wrapping base 902 and attachment tabs 906a and 906b over the exterior of the common carotid artery. The reinforcement tabs 906a or 906b may then be secured to the reinforcement strip 910a, by suturing, stapling, fastening, gluing, welding, or other means. The reinforcement tabs 906c and 906d may be cut off at their bases, as shown at 922 and 924, respectively.

Because various modifications, substitutions, and changes of this invention may be made by one of skill in the art without departing from the spirit thereof, the invention is not limited to the embodiments illustrated and described herein. Rather, the scope of the invention is to be determined by the appended claims and their equivalents.

What is claimed is:

1. An electrode for implantation in biological tissue comprising:
a plurality of electrically conductive nanostructures defining a matrix having a thickness, a proximal side and a perimeter;
an electrically conductive lead in electrical communication with said matrix; and
wherein each of the electrically conductive nanostructures of said plurality of electrically conductive nanostructures are in direct electrical contact with at least one of the other electrically conductive nanostructures of said plurality of electrically conductive nanostructures.

2. The electrode of claim 1 further comprising an electrically conductive base, said proximal side of said matrix being in electrical contact with said electrically conductive base.

3. The electrode of claim 1 wherein at least a portion of said plurality of electrically conductive nanostructures are elongate.

4. The electrode of claim 3 wherein each of the nanostructures of said portion of said plurality of electrically conductive nanostructures that are elongate are characterized by a length and a cross-section substantially normal to said length, said cross-section having an outer peripheral length less than 30-micrometers.

5. The electrode of claim 4 wherein peripheral length defines a minimum peripheral length along said length of said nanostructure.

6. The electrode of claim 1 wherein at least a portion of said plurality of electrically conductive nanostructures are substantially cylindrical.

7. The electrode of claim 1 wherein at least one of said plurality of electrically conductive nanostructures is a nanotube.

8. The electrode of claim 1 wherein said direct electrical contact of said electrically conductive nanostructures is located at or near said proximal side of said matrix.

9. The electrode of claim 8 wherein a portion of said voids comprise nanochambers.

10. The electrode of claim 1 wherein said electrically conductive nanostructures are non-uniform or randomly oriented.

11. The electrode of claim 1 wherein said perimeter of said matrix is in electrical contact with a skirt.

12. The electrode of claim 1 wherein said matrix is divided into at least two segments separated by and in electrical contact with at least one partition.

13. The electrode of claim 1 wherein said nanostructures define a plurality of voids that enable capture of a solution when said electrode is in contact with a target tissue.

14. The electrode of claim 13 wherein said voids pass through said thickness of said electrode and further comprising a suction device operatively connected to said electrode to draw a suction through said voids.

15. The electrode of claim 1 wherein said nanostructures are comprised of a noble metal or a blended alloy.

16. An electrode for implantation in biological tissue comprising:
a base portion having a face;
a lead operably connected to said base portion, the lead including at least one electrical conductor and a corresponding electrical insulation material; and
a plurality of nanostructures wherein at least a portion of said plurality of nanostructures are operably supported on the face of the base portion and at least a portion of the plurality of nanostructures are electrically connected to the at least one electrical conductor,
wherein said plurality of nanostructures are fabricated from a material selected from the group consisting of a noble metal and a blended alloy.

17. The electrode of claim 16 wherein said nanostructures define a plurality of voids that capture a solution when said electrode is in contact with a target tissue.

18. The electrode of claim 17 wherein said voids pass through said thickness of said electrode and further comprising a suction device operatively connected to said electrode to draw a suction through said voids.

19. The electrode of claim 17 wherein said base is conductive and said plurality of nanostructures covers a portion of said base as a coating.

20. The electrode of claim 17 wherein said base comprises an electrical insulator and said at least one electrical conductor is electrically connected to at least one of said plurality of nanostructures.

21. The electrode of claim 16 wherein at least a portion of the nanostructures of said plurality of nanostructures are elongate.

22. The electrode of claim 21 wherein said portion of said plurality of nanostructures that are elongate are substantially normal to said face of said base portion.

23. The electrode of claim 21 wherein each of said plurality of elongate nanostructures are characterized by a length and a cross-section substantially normal to said length, said cross-section having an outer peripheral length less than 30-micrometers.

24. The electrode of claim 23 wherein said peripheral length defines a minimum peripheral length along said length of said nanostructure.

25. The electrode of claim 16 wherein at least a portion of said plurality of nanostructures are substantially cylindrical.

26. The electrode of claim 16 wherein at least one of said plurality of nanostructures is a nanotube.

27. An electrode assembly comprising:
a wrap assembly comprising a base wrapping having an electrode carrying surface;
at least one implantable electrode operably arranged on said electrode carrying surface, said at least on implantable electrode comprising a plurality of nanostructures fabricated from a material selected from the group consisting of a noble metal and a blended alloy; and
an electrically insulated lead conductor in electrical communication with said at least one electrode.

28. A charge injection system comprising:
an implantable pulse generator;
an implantable electrode comprising a plurality of nanostructures fabricated from a material selected from the group consisting of a noble metal and a blended alloy; and
an electrically insulated lead conductor operably connecting said implantable impulse generator to said implantable electrode to deliver an electrical stimulation to a target tissue.

29. The charge injection system of claim 28 further comprising a suction source operably connected to said implantable electrode, said implantable electrode being configured to enable transfer of a suction therethrough.

30. The charge injection system of claim 28 wherein said electrical stimulation delivered by said implantable pulse generator has an enhanced efficiency when delivered through said plurality of nanostructures.

31. A method of mapping the response to baroreflex activation comprising:
selecting a charge injection system comprising an electrode having a distal side and a porous body that enables fluid communication between said distal side and said body;
positioning said electrode at a desired location on a target tissue; and
applying a suction to said porous body to draw said target tissue to said distal side of said electrode.

32. The method of claim 31 further comprising fixing said electrode to said target tissue and releasing said suction.

33. The method of claim 31 further comprising:
(a) measuring the response to baroreflex activation;
(b) releasing said suction to said body;
(c) disengaging said distal side of said electrode from said target tissue;
(d) repositioning said electrode at a different location on said target tissue; and
(e) reapplying said suction to said porous body to draw said target tissue to said distal side of said electrode.

34. A method of injecting charge into a biological tissue comprising:
selecting a charge injection system comprising at least one electrode assembly having a plurality of nanostructures, said nanostructures being fabricated from a material selected from the group consisting of a noble metal and a blended alloy;
placing said at least one electrode assembly in electrical contact with a target tissue; and
applying a charge to said electrode.

35. The method of claim 34 wherein said charge is a voltage charge.

36. The method of claim 35 wherein said voltage charge is greater than 100 millivolts.

37. A method of fabricating a nanostructure electrode comprising:
arranging a plurality of nanostructures to define a matrix having a proximal side, said plurality of nanostructures being fabricated from a material selected from the group consisting of a noble metal and a blended alloy; and
heating at least a portion of said proximal side of said matrix to cause said plurality of nanostructures to fuse together.

38. The method of fabricating of claim 37 wherein said heating is accomplished by irradiating at least a portion of said matrix with a laser.

39. The method of fabricating of claim 37 further comprising:
placing a base portion in contact with said proximal side of said matrix; and
heating at least a portion of said base portion to cause said base to fuse with at least a portion of said matrix.

40. An electrode for injection of a charge into a biological medium comprising:
a charge source;
at least one electrode having a means for enhancing surface area; and
a means for transferring charge from said charge source to said at least one electrode.

41. A charge injection system comprising:
an electrode;
a lead in electrical contact with said electrode; and
a coating of nanostructures disposed on at least a portion of said electrode.

42. The charge injection system of claim 41 wherein said electrode, said lead and said coating of nanostructures are fabricated from a material selected from the group consisting of a noble metal and a blended alloy.

43. The charge injection system of claim 41 wherein said coating of nanostructures is comprised of a matrix of randomly oriented nanostructures.

44. The charge injection system of claim 41 further comprising a suction source operably connected to said electrode, said electrode and said coating being configured to enable transfer of a suction therethrough.

45. The charge injection system of claim 41 further comprising a pulse generator in electrical communication with said electrode.

46. A method for bonding a nanotube to a base electrode comprising:
selecting a nanotube having a first melting point temperature;
selecting a filler material comprising a noble metal or biocompatible blended alloy having a second melting point temperature, said second melting point temperature being lower than said first melting point temperature;
selecting a base;
at least partially filling said nanotube with said filler material to form a filled nanotube;
placing said filled nanotube in contact with said base;
applying an electrical current to said filled nanotube to heat said filler material to a temperature above said second melting point temperature and causing said filler material to flow onto said base; and
cooling said filler material to form a bond between said filler material and said nanotube structure and to form a bond between said filler material and said base.

47. The method of claim 46 further comprising selecting platinum or a platinum iridium alloy of not more than thirty percent iridium as said filler material and selecting nanotubes comprised of iridium.

48. A method of bonding a matrix of nanostructures together comprising:
selecting a plurality of nanostructures having a first melting point temperature;
selecting a filler material comprising a noble metal or biocompatible blended alloy having a second melting point temperature, said second melting point temperature being lower than said first melting point temperature;
assembling said plurality of nanostructures to form a matrix;
packing said matrix with said filler material to form a packed matrix;
heating said packed matrix so that said filler material attains a temperature above said second melting point temperature and causing said filler material to flow; and
cooling said filler material to form a bond between said filler material and said plurality of said nanostructures and to form a bond between said filler material and said nanostructures.

49. The method of bonding of claim 48 further comprising arranging said matrix to form a uniform or a non-uniform matrix.

50. The method of bonding of claim 48 wherein said heating further comprises passing an electrical current through said packed matrix.

51. The method of bonding of claim 48 further comprising selecting platinum or a platinum iridium alloy of not more than thirty percent iridium as said filler material and selecting nanotubes comprised of iridium.

52. An electrode for delivering stimulation comprising:
a matrix formed of a plurality of nanostructures, each of the plurality of nanostructures comprising:
a nanotube formed having a first melting point temperature and having a interior volume defined by the nanotube; and
a filler material comprising a noble metal or biocompatible blended alloy having a second melting point temperature, said second melting point temperature being lower than said first melting point temperature, the filler material filling at least a portion of the interior volume of the nanotube,
wherein said matrix is first heated so that said filler material attains a temperature above said second melting point temperature and below said first melting point temperature and causing said filler material to flow and then cooling said matrix to a temperature below said second melting point temperature to form a bond between said filler material and said plurality of said nanostructures and to form a bond between said filler material and said nanostructures.

53. The electrode of claim 42 wherein said nanotubes are comprised of iridium and said filler material is comprised of platinum.
